(19) United States
(12) Patent Application Publication
(10) Pub. No.: US 2008/0206150 A1
(43) Pub. Date: Aug. 28, 2008

(54) Nanoparticles for Imaging Atherosclerotic Plaque

(75) Inventors: Angelique Y. Louie, Woodland, CA (US); Benjamin R. Jarrett, Davis, CA (US)

Correspondence Address: MORRISON & FOERSTER LLP, 425 MARKET STREET, SAN FRANCISCO, CA, 94105-2482, US

(73) Assignee: The Regents of The University of California, Oakland, CA (US)

(21) Appl. No.: 11/630,822

(22) PCT Filed: Jun. 22, 2005
(86) PCT No.: PCT/US2005/022239

§ 371 Date: Nov. 8, 2007

Related U.S. Application Data
(60) Provisional application No. 60/582,768, filed on Jun. 25, 2004.

(51) Int. Cl. A61K 49/18; A61K 49/00; A61P 9/10; A61K 49/06

(52) U.S. Cl. 424/9.32; 424/9.1; 424/9.3; 424/9.6; 977/773

(57) ABSTRACT

Atherosclerosis is an inflammatory disease of the arterial walls and represents a significant health problem in developed nations. Described is a targeted Magnetic Resonance Imaging (MRI) contrast agent for in vivo imaging of early stage atherosclerosis. Early plaque development is characterized by the influx of macrophages, which express a class of surface receptors known collectively as the scavenger receptors (SR). The macrophage scavenger receptor class A (SRA) is highly expressed during early atherosclerosis. The macrophage SRA therefore presents itself as an ideal target for labeling of lesion formation. By coupling a known ligand for the scavenger receptor, dextran sulfate, to a MRI contrast agent, early plaque formation can be detected in vivo. Targeted MR contrast agents offer a unique opportunity to visualize early plaque development in vivo with high sensitivity and resolution, allowing or early diagnosis and treatment of atherosclerosis.

### CROSS REFERENCE TO RELATED APPLICATIONS

This application claims the benefit of U.S. Provisional Application 60/582,768, filed Jun. 25, 2004 which is hereby incorporated by reference in its entirety.

### FIELD OF THE INVENTION

The compositions and methods described herein generally relate to coated nanoparticles used for the detection of macrophages and inflammatory diseases such as atherosclerosis.

### BACKGROUND OF THE INVENTION

Heart disease is one of the leading killers in developed nations. In the United States alone there are approximately 5 million Americans living with heart disease with 550,000 new cases each year. Furthermore, roughly three quarters of the million cardiovascular disease (CVD) deaths each year are due to atherosclerosis (Heart Disease and Stroke Statistics—2005 Update, American Heart Association: American Heart Association; 2005. 1-63 p.), an inflammatory disease of the arterial vessel wall. Early diagnosis of atherosclerosis would allow for early treatment of the disease, as it is shown to be reversible (Libby et al., 2002, Brown et al, 1993).

The arterial wall is composed of an inner, luminal, endothelial cell layer (intima), a smooth muscle cell layer (media) and an outer layer (adventitia) composed of loose connective tissue and elastin. Atherosclerotic plaque first develops as a lipid deposit between the intima and media at sites of endothelial dysfunction (Ross et al., 1999, Heinecke et al., 1998). Oxidative stress due to poor diet, smoking, irregular flow at bifurcations and stress can lead to endothelial dysfunction and modification of lipids, specifically low-density lipids (LDL). The first immune response to modified LDL build-up is the infiltration of macrophages, which phagocytose the modified LDL in attempt to remove the modified lipid (Glass et al., 2001, de Winther et al., 2000, Ross et al., 1999, Sakai et al., 2000), (FIG. 1). As the macrophages accumulate more lipids they release pro-inflammatory cytokines (Ross et al., 1999, Ross et al., 1993), resulting in an increasing flux of immune cells. Macrophages, activated monocytes and neutrophils also release myeloperoxidase, an abundant heme protein which may play a role in LDL oxidation (Podrez et al, 1999), which may convert more lipids into atherogenic form. Additionally, the macrophages begin to accumulate large amounts of oxidized LDL (oxLDL) and appear foam-like, which macroscopically is seen as a fatty streak (FIG. 1). The plaque further progresses by the accumulation and retention of more immune cells, including T-cells; smooth muscle cells migrate from the media into the lipid core, a necrotic core forms and a fibrous cap forms over the necrotic/lipid core. The plaque can then extend into the lumen and obstruct blood flow, eventually leading to ischemia of distal tissues. Or the fibrous cap can become weakened due to immune cell activity and rupture, forming embolisms that can occlude smaller vessels of the heart or brain, leading to myocardial infarction or stroke, respectively.

The current gold standard for detecting atherosclerosis, angiography, is only capable of detecting stenosis, which yields no information about plaque development within the vessel wall. Both Gd and iron oxide contrast agents have been used in cardiovascular imaging (Ruehm et al., 2001, Jaffer et al., 2004, Winter et al., 2003). Contrast enhanced imaging of atherosclerosis has been performed with the iron oxide particles in both animals and humans, with several in vitro and in vivo (animal models) attempts to increase the specificity of plaque labeling (Jaffer et al., 2004, Choudhury et al., 2002). Angiogenesis has been shown to be associated with plaque development and instability (O'Brien et al., 1994, de Boer et al., 1999) and presents an opportunity for imaging plaque development. Winter and colleagues (Winter et al., 2003b) have shown that αᵥβ₃ (a known marker for angiogenesis) targeted gadolinium particles enhance contrast in atherosclerotic lesions in rabbit aorta. Other developments to target atherosclerotic plaques have been with fibrin-targeted Gd nanoparticles (fibrin is a marker for thrombosis) (Flacke et al., 2001, Winter et al., 2003a), and myeloperoxidase activated iron oxide particles (Perez et al., 2004) or myeloperoxidase activated Gd-chelates (Chen et al, 2004). However, these targeted agents are for markers that are expressed at advanced stages of the disease, not the initial development.

Dextran coated iron oxide particles, such as Feridex, and the smaller ultrasmall superparamagnetic iron oxides (USPIOs) (Schmitz et al., 2000, Ruehm et al., 2001, Schmitz et al., 2002); have been proposed for imaging plaque development. Dextran coated iron oxide particles are nonspecifically taken up by monocytes (immature macrophages) (Schmitz et al., 2000, Ruehm et al., 2001) in circulation and also macrophages confined to the plaque (Schmitz et al, 2001). Magnetic Resonance (MR) images are then acquired after uptake and decreased signal intensity at plaque sites has been observed in animal (Schmitz et al., 2000, Ruehm et al., 2001, Schmitz et al., 2002) and human studies (Schmitz et al, 2001, Kooi et al., 2003). However, large doses of USPIOs are used, approximately 10 times the permitted clinical dose used in animal studies (Schmitz et al., 2000, Ruehm et al., Yancy et al., 2005), as they are cleared by the reticuloendothelial system, particularly the lymph nodes, bone marrow and liver (Schmitz et al., 2000, Bulte et al., 2004, Wilhelm et al., 2003). Current imaging techniques are not sophisticated enough for the detection of early plaque components and early plaque development. A targeted MRI contrast agent for atherosclerosis that would specifically label plaques, would be of great interest clinically to allow detection early enough for successful intervention and treatment of atherosclerosis.

### SUMMARY OF THE INVENTION

The present invention meets these needs by providing a targeted contrast agent for in vivo imaging of atherosclerosis.

Macrophage infiltration at the early development of the disease presents an opportunity for targeted imaging. The macrophage expresses a class of receptors known as scavenger receptor A (SRA), which is primarily expressed on macrophages, but not on normal arterial wall (de Winther et al., 2000). Furthermore, studies have shown (Dejager et al., 1993) that a type of scavenger receptor is also expressed on smooth muscle cells in the developing plaque. Macrophage SRA recognize a broad range of polyanionic molecules, such as oxLDL, polyinosinic acid, fucoidan, dextran sulfate, maleylated-BSA, and silica (de Winther et al., 2000).

The contrast agent of the present invention is coupled to ligands that are recognized by macrophage specific receptors to develop a targeted contrast agent. Since the migration of macrophages into a disease tissue is a dynamic process, utilization of receptors on immune cells enables contrast imaging of the progression of the disease and because of the specificity, enables low doses of contrast agent to be used. The ability to track the progression of the disease with high specificity and low dose (of contrast agent) could lead to a greater understanding of disease progression and aid in development of therapeutics.

In one format, the present invention is directed to a method of imaging a macrophage. The macrophage may express SRA. The method may include contacting a macrophage with a detection agent and detecting the agent to thereby image the macrophage. The detection agent may include a detectable nanoparticle core, a coating and a receptor binding moiety. The receptor binding moiety binds to a receptor on a macrophage. The macrophage may be in a mammalian artery. The macrophage may be in an atherosclerotic plaque. The atherosclerotic plaque may be in a human patient.

The detection agent may be administered by intravenous or intraarterial injection. The detection agent may be a magnetic resonance imaging agent or a fluorescence spectroscopy agent.

In one format, the detectable nanoparticle core is a metal oxide or a doped semiconductor. The metal oxide may be an iron oxide, a manganese oxide or a lanthanide oxide. The doped semiconductor may be doped with a paramagnetic atom or a paramagnetic molecule. The nanoparticle core may be a CdS or a ZnS nanoparticle. The nanoparticle core generally has a dimension less than about 100 nm. The range of the particle size is between about 1 nm and about 30 nm, between about 4 nm and about 15 nm and between about 8 nm and about 12 nm.

The coating may be a polymer coating. The coating may be dextran sulfate or silica. The coating may also be a receptor binding moiety. The receptor binding moiety may be polyanionic.

The receptor binding moiety may be covalently attached to a linker molecule attached to the nanoparticle core. The linker molecule may be a polyethylene glycol derivative. In one format, the linker molecule has a first functional group capable of binding to the nanoparticle core and a reactive functional group for attachment to the receptor binding moiety.

In another format, the receptor binding moiety may be an anionic moiety such as oxLDL, polyinosinic acid, fucoidan, dextran sulfate, or maleylated-BSA.

The invention is further directed to an imaging agent including a detectable nanoparticle core a coating, a receptor binding moiety and a secondary detection moiety. The core may be detectable by magnetic resonance imaging. The core may be an iron oxide, a manganese oxide, a lanthanide oxide or a semiconductor doped with a paramagnetic atom or molecule.

In one format, the secondary detection moiety is a fluorescent detection moiety or a positron emitting detection moiety. The secondary detection moiety may include ⁶⁴Cu. The nanoparticle core may be fluorescent such as a CdS or a ZnS nanoparticle. The secondary detection moiety may be a magnetic resonance imaging contrast agent or a PET detection moiety.

The coating may be a polymer coating. The coating may be dextran sulfate or silica. The coating may also be a receptor binding moiety. The receptor binding moiety may be polyanionic.

In another format, the invention is further directed to a composition for imaging. The composition may include a detectable nanoparticle core a coating and a receptor-binding moiety. The receptor-binding moiety may be polyanionic such as oxLDL, polyinosinic acid, fucoidan, dextran sulfate and maleylated-BSA. The core may be detectable by magnetic resonance imaging. The core may be an iron oxide, a manganese oxide, a lanthanide oxide or a semiconductor doped with a paramagnetic atom or molecule.

The coating may be a polymer coating. The coating may be the receptor binding moiety. The receptor binding moiety may be covalently attached to a linker molecule attached to the nanoparticle core. The linker molecule may be a polyethylene glycol derivative. The linker molecule may have a first functional group capable of binding to the nanoparticle core and a reactive functional group for attachment to the receptor binding moiety. The receptor binding moiety may be an anionic moiety such as oxLDL, polyinosinic acid, fucoidan, dextran sulfate, or maleylated-BSA.

The present invention is further directed to a method for producing a dextran sulfate coated nanoparticle. In one embodiment, a solution of diphenyl ether, 1.2-hexadecandiol, oleic acid, oleylamine and iron acetylacetate is heated. The heating can be performed at about 300 degrees centigrade to form the iron oxide core. Dextran sulfate can then be attached to the prefabricated core by electrostatic absorption. A second method may include heating a solution of iron chloride in the presence of reduced dextran and dextran sulfate. For example, the reduced dextran can be present in about 10 to about 100 times the concentration of the dextran sulfate.

### BRIEF DESCRIPTION OF THE DRAWINGS

FIG. 1 shows the development of atherosclerotic plaque.

FIG. 2 shows Scheme 1, a synthetic protocol for Dextran Sulfate coated Iron oxide particles using an absorptive layering technique. A shows formation of iron oxide core stabilized by oleic acid and oleylamine. B shows transfer of iron oxide core to water and subsequent absorption of Dextran Sulfate onto particle surface.

FIG. 3 shows TEM of 85 nm Dextran Sulfate coated iron oxide. Core diameter is 60 nm and coating is about 7 nm in thickness.

FIG. 4 shows Scheme 2, the synthesis of Silica coated particles by a base hydrolysis of TEOS and subsequent absorption of Silica onto iron oxide surface.

FIG. 5 shows TEM showing 10 nm iron oxide core and silica coating.

FIG. 6 shows TEM of 6 nm iron oxide cores coated with silica. Overall diameter by dynamic light scattering is 52 nm.

FIG. 7 shows P388D1 cell study demonstrating selective uptake of silica nanoparticles. A shows a MR image of agar suspensions of cells after incubation (according to table 1). A decrease in signal intensity is seen in samples 2-5, with a return to control signal in sample 6. B shows the mean signal intensity (±standard deviation) of a circular region of interest in A is plotted for each sample. A general trend of signal intensity decrease with particle uptake and followed by increase in intensity by competition with dextran sulfate is seen.

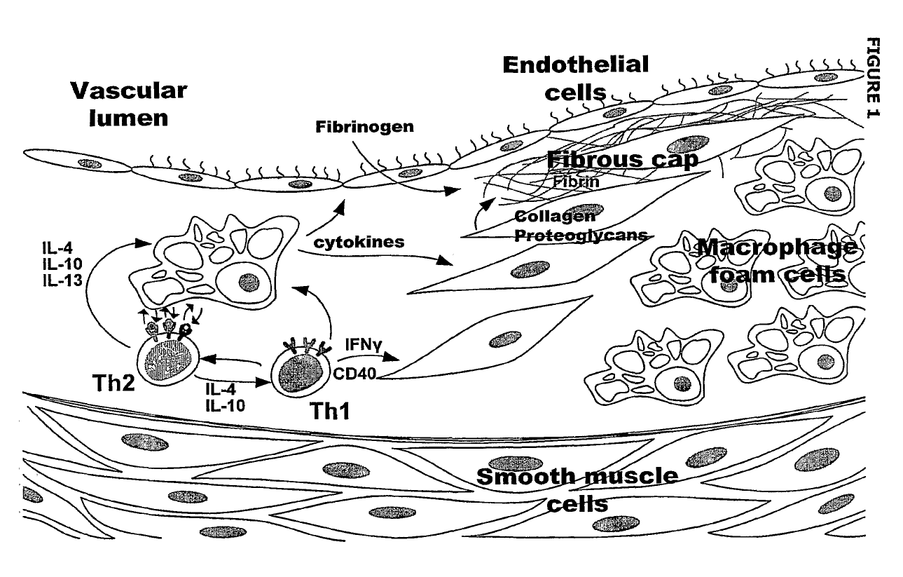

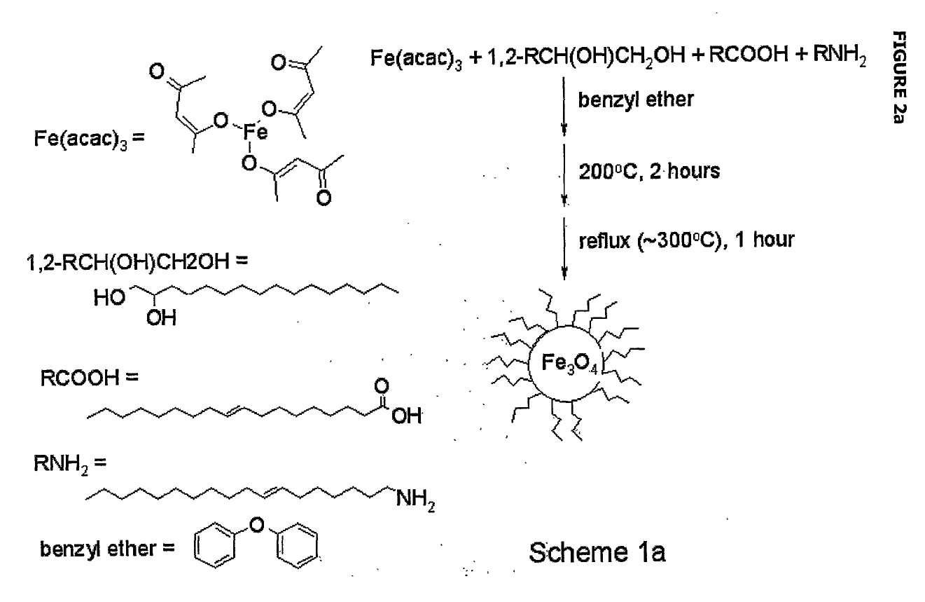

### DETAILED DESCRIPTION OF THE INVENTION

### Principles of Magnetic Resonance Imaging

Magnetic Resonance Imaging is widely used clinically because it is non-invasive, non-ionizing, and offers excellent soft tissue contrast. Certain nuclei, including ¹H, ¹³C, ²³Na, ³¹P, possess a net nuclear spin. These spins, when placed in a strong external magnetic field can either align with or against the main field. Magnetic Resonance Imaging (MRI) is based on the principle that a slight excess of these spins will align with the main field, Bₒ. The net excess of spins in a voxel (volume of space), which can be collectively thought as a magnetic moment, aligned with Bₒ precesses about the main field direct at the Larmor Frequency, ω_{L}=γBₒ. Where ω_{L} is the Larmor frequency, γ is the gyromagnetic ratio of the proton, and Bₒ is the main filed strength. If a 90° radiofrequency, RF, pulse is applied at ω_{L} the magnetic moment will be tipped into the plane perpendicular to the main field, the transverse plane. The precession of the magnetic moment in the transverse plane generates a magnetic flux, which, by Faraday's law, can induce a voltage in a loop of wire. The same coil that generates the RF pulse is often used for receival of this oscillating magnetic moment, and this is the MR signal. Since the magnetic moment oscillates only at the Larmor frequency one can control which collection of spins are perturbed and also know exactly what frequency, the Larmor frequency, to record.

In MRI the proton, ¹H, is primary used due to its strong gyromagnetic ratio and natural abundance, as the human body is approximately 70% water. Once the RF pulse is removed the magnetic moment will begin to relax back to an equilibrium state, by two independent mechanisms, longitudinal (T1) and transverse (T2) relaxation. Longitudinal relaxation is the recovery of magnetic moment along the main field direction. T1 is also known as the spin-lattice relaxation rate, as this process occurs by the transfer of energy between proton spins and the surrounding lattice. The lattice can be thought of as the thermal pool of energy the spins are originally in equilibrium with. On the other hand, the transverse relaxation, or spin-spin relaxation, is the transfer of energy between spins of the protons, and this results in dephasing of the transverse component (as the magnetization moment is just an ensemble of spins) of the magnetization. An additional term can also be introduced, T2*, which is T2 decay (the random spin-spin interaction) plus dephasing due to magnetic field inhomogeneity; T2* is always shorter than T2. The MR signal is therefore a combination of T1, T2, T2*, and proton density (N(H), more protons equals more signal).

### MR Image Generation

The above discussion describes the process by which an MR signal is generated, however it does not account for the spatial information of the MR image. Spatial information is generated by applying linear gradients, which results in different Larmor frequencies at different locations in the object. The linear change in Larmor frequencies generates unique frequency components that can be converted (one-to-one because linear) to unique spatial locations in the MR image with the Fourier Transform, which mathematically relates the frequency and spatial domains. An RF pulse is applied to a slice or slab, of selected frequencies of interest, to excite proton spins in a volume of space. Gradients are applied in two or three dimensions along the slice, encoding spatial information, to generate 2 or 3D images, respectively. Contrast in MR images is primarily due to the tissues intrinsic relaxation rates, 1/T1 and 1/T2. Pulse sequences that favor 1/T1 or 1/T2 are then implemented by adjusting image parameters to weight the signal intensity to reflect 1/T1, or 1/T2 differences. As different tissues have significantly different T1 and T2 values, MRI offers excellent tissue contrast. It has been shown (Kramer et al., 2004, Toussaint et al., 1996, Toussaint et al., 1995, Herfkens et al., 1983, Rogers et al, 2005) that the components of atherosclerotic plaque can be imaged using a combination of proton density weighted (PDW), T1-weighted (T1W) and T2-weighted (T2W) scans.

### Contrast Enhanced MRI

Although conventional MRI offers excellent soft tissue contrast, it is often inefficient for small structures and cannot distinguish normal tissue from cancerous or diseased tissue that is not enlarged (e.g. due to inflammation). Exogenous contrast agents, which affect T1 and T2 relaxation times of surrounding tissues (or more correctly surrounding protons), can be used to enhance tissue contrast which may not be resolved with typical MR imaging. Contrast enhanced MRI has become a significant diagnostic tool, it composes roughly 30% (Caravan et al., 1999) of all clinical MR images. Two classes of MR contrast agents used clinically are gadolinium-chelates, such as gadolinium(Gd)-DTPA and Gd-HP-DOTA, and dextran coated iron oxide particles. The Gd-DTPA (and Gd-HP-DOTA) acts primarily as a positive or T1 contrast agent, as it decreases the T1 time of surrounding protons, resulting in an increased signal intensity on T1W images. The Gd³⁺ ion is paramagnetic; it has 7 unpaired electrons and a strong positive magnetic susceptibility (ability to become magnetized in a magnetic field). Relaxation of water molecules by Gd³⁺ occurs by direct contact (dipolar interactions) of the water molecules with the paramagnetic ion or through space, although this effect decreases as 1/r⁶, where r is the distance from the paramagnetic ion (Caravan et al., 1999, Lauffer et al., 1987). On the other hand, iron oxide nanoparticles are typically termed negative or T2 contrast agents, as the strong positive magnetic susceptibility results in a rapid dephasing of proton spins and thus a decreased T2 time and a decrease in signal intensity on T2W images. As opposed to the paramagnetic ion chelates (e.g. Gd-DTPA), iron oxide particles have thousands of unpaired electrons, which generate a small magnetic field around the particle. As the water molecules diffuse through the magnetic field generated by the iron oxide particles, their magnetic spins rapidly become dephased, and thus decrease T2 times. This microscopic magnetic field extends beyond the surface of the iron oxide particles, such that these particles can appear up to 50 times larger than the diameter of the particle (Dodd et al, 1999), which enables minute concentrations, μmol to nmol depending on pulse sequence, of contrast agent to be detected (Bulte et al., 2004, Heyn et al., 2005).

### Nanoparticles

One example of a nanoparticle that may be used is a Fe₃O₄ nanoparticle coated with a dextran sulfate coat. Such coated nanoparticles are detectable using MRI imaging techniques (or other magnetic resonance techniques) and will bind to scavenger receptor class A (SRA) receptors expressed on the surface of macrophages. The coated nanoparticles may therefore be used to detect macrophages expressing SRAs and may be used to detect diseases such as atherosclerosis in which SRAs are highly expressed. Details of such Fe₃O₄ nanoparticle coated with dextran sulfate and their use in detecting atherosclerosis is described in detail in the Examples. Also described is a silica coated iron oxide nanoparticle.

The coating of nanoparticles is limited by the yield of product and the attachment of dextran sulfate to the particle surface. The transfer of the oleic acid/oleylamine stabilized particles to water (Euliss et al., 2003) is very dilute and the yield is low due to aggregation. One way to covalently attach dextran sulfate to aminated iron oxide particlesis by first coating the oleic acid/oleylamine particles with amine-PEG (amine-polyethylene glycol) (Nitin et al., 2004) or silyl amine and then attaching the dextran sulfate covalently to the amine groups. Covalent attachment of the dextran sulfate to the amine group, rather than electrostatic absorption using the layering technique, may allow smaller cores to be coated. Pre-coating the iron oxide surface before addition of the dextran sulfate may avoid cross bridging and aggregation seen with the layering technique by removing the high affinity of the sulfate group for the iron core. Furthermore, use of a very thin layer of amine-PEG may allow very small diameter particles to be synthesized.

Generally the coated nanoparticles comprise a nanoparticle core that may be detected using some detection technique and that is coated with some receptor binding moiety capable of binding to a receptor. If the receptor is expressed in cells associated with some disease or condition, such coated nanoparticles may be used to detect the disease or condition.

Below is described in more detail specific coated nanoparticles that may be used in the compositions and methods described herein, and we then describe general nanoparticle core materials, physical dimensions of nanoparticles, receptor binding moieties, cells that may be detected, diseases that may be detected, and detection methods that may be used in the compositions and methods described herein. We also describe possible therapeutic uses of coated nanoparticles, modes of delivery, and formulations of the coated nanoparticles.

Size of Nanoparticles

Generally the size of coated nanoparticles that may be used are any sizes such that the coated nanoparticles may bind to the receptor and may be detected. In one version the coated nanoparticles are approximately spherical and have a diameter of between about 1 nm and about 100 nm. In one version the coated nanoparticles have a diameter of less than about 30 nm. The coated nanoparticles are not limited to spherical nanoparticles. The size of the coated nanoparticles may affect immune detection of the particles and uptake mechanism by cells. Larger coated nanoparticles may also be subject to non-specific phagocytosis.

Generally the size of nanoparticles cores that may be used are any sizes such that the coated nanoparticles comprising the core may bind to the receptor and may be detected. In one version the nanoparticle core is approximately spherical and has a diameter of between about 1 nm and about 30 nm. In another version, the nanoparticle core has a diameter of between about 4 nm and about 15 nm. In another version, the nanoparticle core has a diameter between about 8 nm and about 12 nm.

Generally smaller nanoparticle cores are preferred since small particles have increased relaxation rates and higher signal intensity. Larger nanoparticle cores may become ferromagnetic. Ferromagnetic materials can have very large signals themselves often distorting the image as a result of aggregation of the material. Both paramagnetic and superparamagnetic materials can be used in the nanoparticles, although relaxation rates are better for the superparamagnetic materials.

Nanoparticle Core

When MRI and similar magnetic detection techniques are used, generally the nanoparticle core may be made of any material that renders that coated nanoparticle detectable using MRI. Suitable materials include but are not limited to metal oxides, including iron, manganese and lanthanide oxides, and semiconductors doped with MRI active atoms, molecules or moieties.

Nanoparticle cores that may be used with other detection techniques are described in the “Detection Techniques” section below.

### Receptor Binding Moieties

Generally any receptor binding moiety may be used that is capable of binding to a target receptor. For scavenger receptors present on macrophages dextran sulfate may be used. Additional moieties that may be used include but are not limited to fucoidan, polyguanylic acid, polyinosinic acid, inosine monophosphate, maleylated BSA, acetylated LDL, oxidized LDL, maleylated dextran, acetylated dextran. Other moieties that may be used include but are not limited to polyanionic ligands, oxidized lipids, and poly AA. Receptor binding moieties are also referred to herein as “ligands” and the terms are used interchangeably.

### Cells That May be Detected Using Nanoparticles

Cells expressing scavenger receptors such as SRA may be detected using the compositions and methods described herein. Macrophages are the main cells expressing scavenger receptors and macrophages that may be detected include but are not limited to Kupffer cells, alveolar, spenic, and thymic macrophages. SRA are also expressed on endothelial cells lining the liver and adrenal sinusoids and of endothelial cells of the lymph nodes. There are also some SRA found on the retinal pigment epithelium (eye), so it may be possible to detect inflammatory disease of the retina. The expression of the receptor is variable depending on stimulus and local environment.

The main functions of the SRA, outside of atherosclerosis, are innate immunity and adhesion. It may be possible to detect macrophages in response to inflammation (adhesion events).

### Diseases That May be Detected Using Nanoparticles

Diseases that are characterized by an influx of macrophages or in other ways involve macrophages may be diagnosed using the compositions and methods described herein. In addition to inflammatory conditions such as atherosclerosis, diseases and conditions that may be detected include but are not limited to infections, arthritis, and leukemia.

One example of a disease which could be detected is restenosis, the re-narrowing of a coronary artery after it has been treated with angioplasty or stenting. While the short-term success rate for percutaneous coronary intervention (PCI) in the treatment of vascular occlusions exceeds 90%, 30-60% of patients experience restenosis within six months following a procedure. The use of stents in conjunction with PCI brings the rate of restenosis down to 20-40%, however the rate of restenosis remains undesirably high. The understanding of the mechanisms driving restenosis remains incomplete. The recent development of drug eluting stents shows promise but much needs to be determined regarding the best targets to prevent restenosis. Elucidating the cellular and molecular driving forces in restenosis will help us to develop preventative measures.

Restenosis is now understood to involve a combination of vascular remodeling and intimal hyperplasia; but stenting virtually eliminates the contribution from remodeling. Proliferation of smooth muscle cells is a key step in intimal hyperplasia and a number of drug-eluting stents are directed at prohibiting smooth muscle cell growth, however these have met with inconsistent success. Two FDA-approved drug-eluting stents coated with the immune suppressants Sirolimus (rapamycin) or paclitaxel, have shown a high degree of efficacy in preventing clinically significant restenosis. Accumulation of macrophages is known to be an initial step in restenosis and there may be a connection between the accumulation of macrophages and the activation of smooth muscle cell proliferation but this is difficult to study in vivo. Described herein are nanoparticles for in vivo imaging that will allow us to assess the degree of macrophage recruitment around stents and correlate that accumulation with subsequent restenosis.

### Detection Techniques

As described, MRI may be used to detect the coated nanoparticles. Other detection techniques that may be used include but are not limited to positron emission tomography (PET), optical detection, and detection of radiolabeled particles. When PET is used, the nanoparticle is detectable by PET and when optical detection is used the nanoparticle is detectable by optical detection.

Examples of PET detectable coated nanoparticles include but are not limited to the following: (1) Dextran sulfate coated iron oxide nanoparticle in which the dextran sulfate has been functionalized to allow attachment of chelated (e.g. DOTA) positron emitter (e.g. Cu-64); and (2) coated metal oxide nanoparticle which is subjected to neutron beam bombardment, for neutron beam radiography.

Examples of optically detectable coated nanoparticles include but are not limited to fluorescent nanoparticles, including CdS and ZnS nanoparticles.

Also contemplated are combinations of the above detection techniques. For example, it is contemplated to create a nanoparticle which is detectable by both MRI for in vivo work, and by fluorescence microscopy for histological studies.

It is highly desirable to combine the sensitivity and temporal resolution of PET with the high resolution anatomical information of MRI. A dual MRI/PET contrast agent targeted to atherosclerosis would allow for easy detection of contrast agent uptake (PET) and anatomical detail of lesion development (MRI). For example, Amine-PEG iron oxide nanoparticles can be coupled to p-NCS-benzyl-DOTA, a metal chelator, to carry the PET agent 64Cu. These particles can also be conjugated to dextran sulfate, the targeting moiety for macrophage Scavenger Receptor (SR) and atherosclerosis. This creates a targeted agent which can be detected by both MRI and PET.

### Therapeutic Uses

The compositions described herein may be used for therapeutic uses. For example, it may be possible to include a therapeutic core or coating. For example, the compositions may be able to deliver therapeutic doses of radiation. It may be possible to use Cu64-DOTA sub Cu67 with other nuclides. Cu64 can be used as both a PET agent (imaging) and therapeutic agent. Cu64 emits both positrons, which are used in PET imaging, and beta particles, which can be used for therapy. Other nuclides are used for therapy because they decay primarily by beta emission, whereas most clinical PET agents decay primarily by positron emission.

### Administration and Formulations of Coated Nanoparticles

Formulations containing the coated nanoparticles may be administered by any method capable of delivering the coated nanoparticles to the required tissue and cells. For example, the formulation may be administered intravenously. Other routes of administration that may be used include but are not limited to inhalation of an aerosol formulation of coated nanoparticles and oral administration of a solid dosage form.

Generally, any formulation of coated nanoparticles may be used that is capable of being administered to a subject. Formulations that may be used include but are not limited to liquid formulations, solid formulations and aerosol formulations.

Dried polysaccharide (e.g. dextran sulfate) coated particles may aggregate. For storage it may therefore be preferable to mix the particles in a salt solution (for example, phosphate buffered saline) and then dry the particles to prevent aggregation.

### Subjects That May be Treated or Diagnosed

Generally the compositions and methods described herein may be used for diagnosing or treating diseases or conditions in any subject, including but not limited to human subjects and non-human mammal subjects, such as farm animals or pets. A preferred subject is a human subject.

The invention will be better understood by reference to the following non-limiting examples.

### EXAMPLE 1

### Dextran Sulfate Coated Iron Oxide Nanoparticles Created by Layering

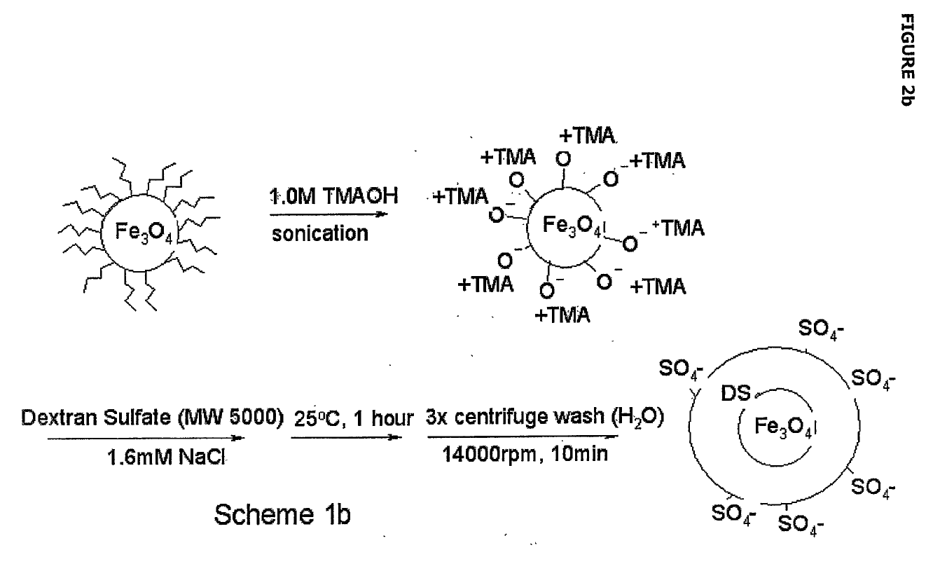

The iron oxide core was synthesized using a method by Sun and colleagues (Sun et al., 2004) for synthesis of oleic acid/oleylamine coated particles that allows for precise control of particle size. Control of particle size is useful for modeling relaxation properties of the particles and tailoring optimal contrast agent design, as relaxation is size dependent (Yung et al., 2003, Koenig et al., 2002, Roch et al., 1999, Koenig et al., 1995). The general synthesis is shown in scheme 1 (FIG. 2), in which the iron oxide core is formed, transferred to water, and then coated with dextran sulfate via a layer-by-layer (LbL) technique. Magnetite cores were formed using a protocol by Sun (Sun et al., 2004) in which an iron precursor is oxidized to form 6 nm iron oxide. The oleic acid/oleylamine stabilized iron oxide particles were then transferred to water using tetramethylammonium hydroxide (TMAOH) (Euliss et al., 2003). The cores were then coated with dextran sulfate using a LbL technique (Gittins et al., 2000, Gittins et al., 2001) in which a charged sphere is coated with a polymer with opposite charge by electrostatic absorption. By using an appropriate salt concentration a polymer may become flexible enough to overcome the sharp radius of curvature of a small sphere and wrap around the core (Gittins et al., 2001, Netz et al., 1999). By further choosing an appropriate polymer length, one that is short enough to avoid the ends from contact upon coating (e.g. polymer length less than circumference of sphere) and not so short that the core sphere is insufficiently coated (which would promote aggregation via cross linking of multiple cores) the iron oxide cores can be coated with dextran sulfate. With 1.6 mM NaCl and 5000 MW dextran sulfate, the 60 nm iron oxide cores were coated with dextran sulfate.

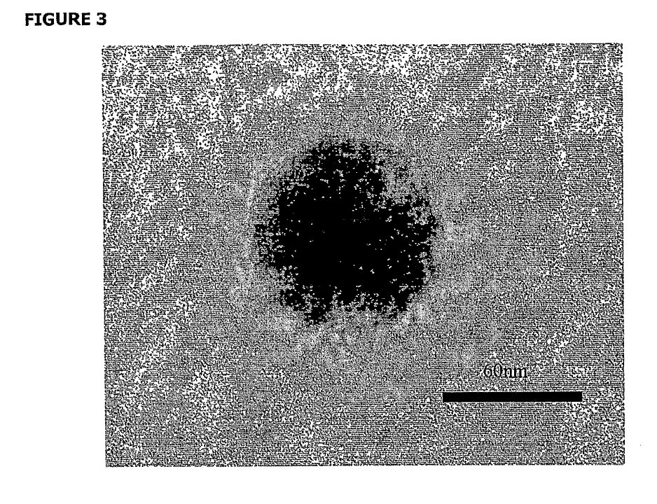

A TEM image of 85 nm particles, with a 60 nm iron oxide core, is shown in FIG. 3. Dynamic light scattering showed a hydrodynamic radius (radius of the particles in solution) of 85 nm, confirming TEM measurements.

### EXAMPLE 2

### Dextran Sulfate Doped Iron Oxide Nanoparticles

The initial dextran coated particle synthesis (Palmacci et al., 1993, Paul et al., 2004) was altered to include a small amount of dextran sulfate mixed with reduced dextran (rd) to form DS-doped-rdUSPIOs. Smaller particles less than 50 nm may be ideal as their smaller size will increase circulation time and reduced clearance by the reticuloendothial system (Pratten et al., 1986, Bowen et al, 2002).

We modified the USPIO synthesis proposed by Paul and colleagues (Paul et al., 2004) to include a small proportion (˜5%) of dextran sulfate mixed in with the reduced dextran. The general core synthesis is as follows: FeCl₂+2FeCl₃→[Fe(OH)₂+2Fe₂O₃.dextran (or dextran sulfate)]→Fe₃O₄.dextran/dextran sulfate; where the brackets represent an intermediate step (Thomassen et al., 1991). Using a very small percentage of dextran sulfate may allow the iron oxide cores to form with a small amount of sulfate groups attached for recognition by the macrophage SR. The DS-doped rdUSPIO particles had a mean hydrodynamic diameter of 88 nm by dynamic light scattering and a core diameter of 6±2 nm, determined by TEM. Furthermore, sulfate content was qualitatively shown using a toluidine blue assay (Aaraki et al., 2004), demonstrating particles have some dextran sulfate content after purification. Interestingly, there were two distinct size populations observed by DLS; one centered at 30 nm and a larger population centered at 100 nm.

### EXAMPLE 3

### Silica Coated Iron Oxide Nanoparticles

Synthesis

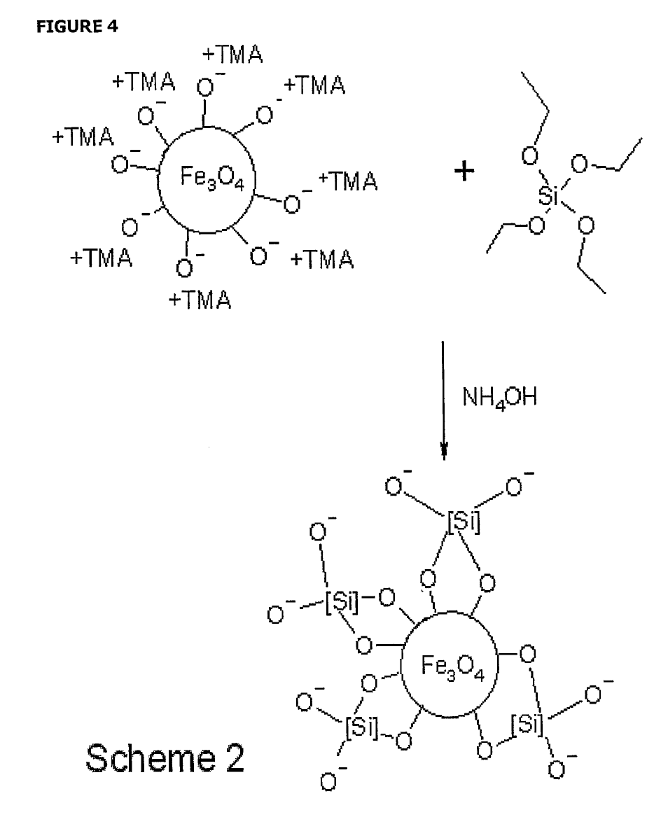

Silica coated particles were synthesized. Silica particles have been widely used for stabile nanoparticles platforms as they are stabile in a wide pH range (Klotz et al., 1999) and silica, due to its polyanionic nature, has been shown to be recognized by the macrophage SR (Platt et al., 2001). We show here that silica coated particles are recognized by macrophages and can be used to label atherosclerotic plaques. The general synthetic route is shown in scheme 2 (FIG. 4). The iron oxide cores were again synthesized according to the Sun protocol (Sun et al, 2004), and transferred to water as before (Euliss et al., 2003). Silica coated particles were then made by the absorption of Si onto the iron oxide by base hydrolysis of tetraethylorthosilicate (TEOS) (Lu et al., 2002).

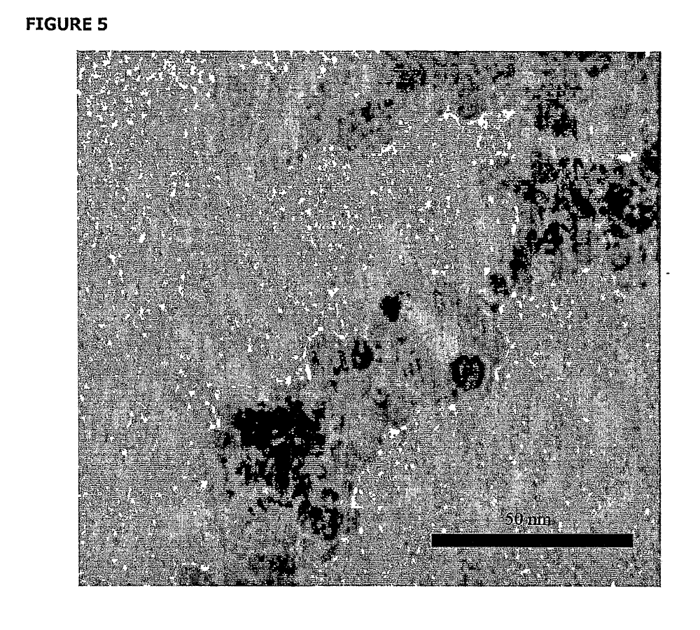

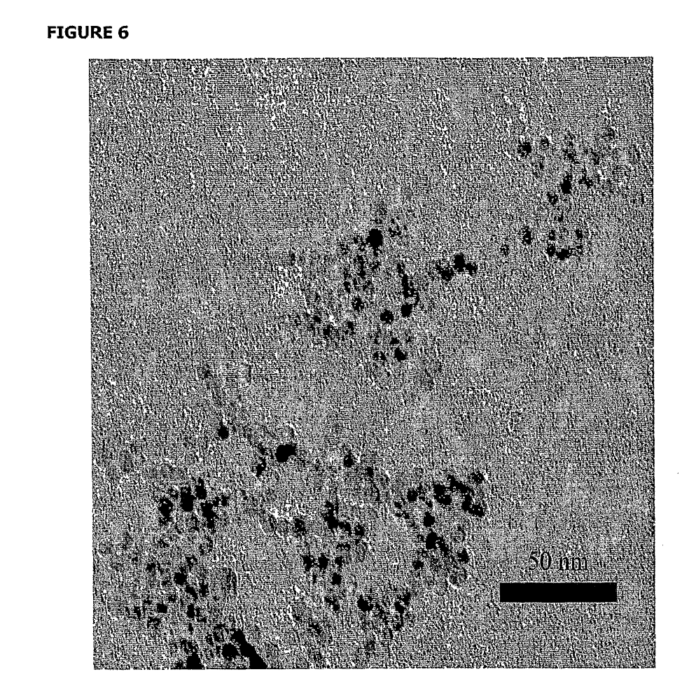

FIG. 5 is a TEM image of 80 nm Silica coated particles demonstrating a 10 nm iron oxide core. Dynamic light scattering showed an overall particle diameter of 80 nm. A second silica coated particle was created by adjusting the amount of base. This second coated particle has a size of 52 nm with a 6 nm iron oxide core. (FIG. 6).

However, the bare SiO₂ coated particles are unstable and precipitate over time in aqueous solutions. Furthermore, the dynamics in salt and or protein solutions are not understood, as the silica particles aggregate rapidly in salt solutions, but much slower in protein solutions freshly prepared. These particles could be used quickly before aggregation, or the particles could be modified to increase their stability in solution.

Verification of Receptor Mediated Uptake.

The 80 nm Si—Fe₃O₄ particles (in water) were 0.2 μm syringe filtered and RPMI (10% FBS and L-glutamine) was added to yield a 1.51 mM Fe particle solution. P388D-1 macrophages were used and were cultured with RPMI (10% FBS and L-glutamine) at 37° C. and 5% CO2. P388D-1 cells were in 35 mm cell culture dishes (Falcon, 353001) at approximately 11.75×10⁴ viable cells/mL. P388D-1 cells were incubated with Si—Fe₃O₄ particles for 1 hour with varying concentrations of a binding competitor, dextran sulfate (0-100 μg), as described in Table 1. The competition study is based on receptor access. If a large excess of competitor ligand for the scavenger receptor is present in the culture media with the particles, the probability of a receptor binding the competitor instead of the particles increases. However, if receptors are not the primary mechanism for particle binding the uptake by macrophages is then non-specific phagocytosis, which is not mediated by receptors. Because the plasma membrane is continuously turning over, the number of “sites” for non-specific phagocytosis is infinite and increasing the concentration of a competitor, dextran sulfate, will not inhibit particle uptake.

TABLE 1

| | particle | dextran sulfate |
|---|---|---|
| | concentration | concentration |
| dish # | (mM Fe) | (μg) |
| 1 | 0 | 0 |
| (control) | | |
| 2 | 1.447 | 0 |
| 3 | 1.447 | 25.6 |
| 4 | 1.447 | 51.2 |
| 5 | 1.447 | 76.8 |
| 6 | 1.447 | 102.4 |

MRI

After 1 hour incubation with Si—Fe₃O₄ particles and binding competitor (dextran sulfate), cells were washed three times with cold RPMI media and then scraped from the dish and centrifuged (1300 rpm, 5 minutes). Cell pellets were resuspended in 500 μL of RPMI and 250 μL melted agar was then added (final 0.35 wt % agar). Particle loaded cells in agar were quickly added to 5 mm NMR tubes and MRI experiments were performed at 21° C. on a Biospec 7T system (Bruker, Billerica, Mass.) equipped with a micro-gradient set, 950 mT/m maximum gradient, and 35 mm ID volume coil. A T2*W FLASH sequence was used with TR/TE/FA/NEX=177 ms/15 ms/15 o/4, matrix size 256×256, FOV 3.2 cm×2.4 cm.

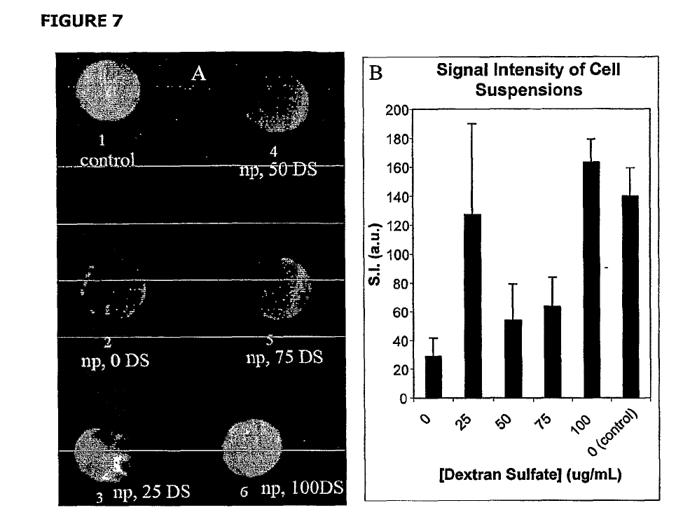

A decrease in signal intensity was seen upon incubation of macrophages with Si—Fe₃O₄ particles (FIG. 7).

The Si—Fe₃O₄ particles are therefore taken up by P388D-1 macrophages. Furthermore, the addition of dextran sulfate to the samples resulted in an increase in signal intensity, demonstrating that the silica particles could be competed out (FIG. 6). This competitive binding supports a receptor mediated endocytic pathway for silica coated particles, since the scavenger receptor (type IIa) recognizes the negative charge of several polyanionic species such as silica, dextran sulfate, poly I, and oxidized LDL.

### EXAMPLE 4

### Reduced Dextran Coated Iron Oxide Particles (rdUSPIO)

Dextran coated particles were synthesized. We began with the one-pot synthesis by Palmacci and colleagues (Palmacci et al., 1993) and obtained 100 nm particles, as determined by dynamic light scattering. We next synthesized dextran coated particles using reduced dextran according to Paul and colleagues (Paul et al., 2004). The reduced dextran method was used in place of the original one-pot synthesis (Palmacci et al., 1993) since it was shown that 20 nm particles could be obtained with a lower excess concentration of reduced dextran compared to unmodified dextran (Paul et al., 2004). The general core synthesis is similar to the dextran sulfate doping technique and is as follows: FeCl2+2FeCl3→[Fe(OH)2+2Fe2O3.dextran]→Fe₃O₄.dextran; where the brackets represent an intermediate step (Thomassen et al., 1991). The rdUSPIO particles had a mean hydrodynamic diameter of 44 nm by dynamic light scattering and a core diameter of 5±1.2 nm, determined by TEM. The 44 nm particle size was polydisperse (wide size distribution) and the mean diameter was larger than the 20 to 30 nm diameter reported by Paul and colleagues (Paul et al., 2004).

### EXAMPLE 5

### Magnetic Properties

Procedures

Particle Size Measurements. Iron oxide core diameter size was determined by Transmission Electron Microscopy (TEM) using a Phillips CM120 at 80 kV. A 5 μL drop of dilute particle sample (approximately 0.04 to 0.4 mM Fe) was put onto the Formvar side of a 300 mesh carbon coated copper grid (Ted Pella #01820) and allowed to air dry before imaging. The hydrodynamic diameter of the coated particles was determined by Dynamic Light Scattering (BI9000AT, Brookhaven). Particle samples for DLS were prepared by diluting particle suspensions to less than 2 mg/ml particle concentration.

Magnetic Measurements. Characterization of magnetic resonance properties of the iron oxide particles was achieved by NMR relaxivity. MRI experiments were performed at 21° C. on a Biospec 7T system (Bruker, Billerica, Mass.) equipped with the standard gradient set, 95 mT/m maximum gradient, and 72 mm ID volume coil. Particle suspensions in deionized water with iron concentration between 0 and 0.4 mM Fe were used. T1 was measured using a sequence of Spin Echo images with independently varying Recovery Times (10 data points, TR, 150-400 ms). T2 was measured using a sequence of Spin Echo images with independently varying Echo Times (8 data points, TE, 6.9-250 ms). Image reconstruction consisted of linking the images together (both T1 and T2 data) and fitting an exponential curve to the data points to determine T1 and T2 for each sample (water and 4 iron concentrations). Circular regions of interest (ROIs) were drawn within the tube cross-sections; the high intensity edges representing the glass tube were not included in the ROIs. Image reconstruction was done in Paravision version 3 (Bruker, Billerica, Mass.). The longitudinal (r1) and transverse (r2) relaxivity were determined as the slope of the line for plots of 1/T1 or 1/T2, respectively, against increasing iron concentration with a correlation coefficient greater than 0.90 (Microsoft Excel 2003).

Results

Table 2 summarizes the magnetic properties, along with particle size of particles synthesized, with a literature value for comparison. The r1 value represents longitudinal relaxivity and the r2 value represents the transverse relaxivity of the particles, a measure of the relaxation rate normalized to iron content, expressed as (sec•mM-Fe)-1. Relaxivity is a measure of how effective a contrast agent affects T1 and T2 relaxation rates, and a larger number indicates a stronger effect. Relaxation rates for the silica particles are comparable with current SPIO particles. An increase in r2 is seen with increasing core size for the nanoparticles, except for the 60 nm core of the DS np: layering particles. The lower relaxivity values of the DS np: layering particles could be due to a different form of iron oxide, which may have a smaller magnetic susceptibility and thus decreased relaxation effect, of the larger cores compared with the smaller cores of the other particles.

TABLE 2

| | | core | r₁ | r₂ |
|---|---|---|---|---|
| contrast agent | diameter | diameter | (mM⁻¹sec⁻¹)* | (mM⁻¹sec⁻¹)* |
| DS np: layering | 80 nm | 60 nm | 0.01 | 52.5 |
| DS np: doping | 88 nm | 6 nm | | |
| SiFe₃O₄ | 80 nm | 10 nm | 1.47 | 278 |
| SiFe₃O₄ | 52 nm | 6 nm | 2.75 | 241 |
| rdUSPIO | 44 nm | 5 nm | 1.57 | 125 |
| literature | | | | |
| value for | | | | |
| comparison**: | | core | r₁ | r₂ |
| contrast agent | diameter | diameter | (mM⁻¹sec⁻¹):+ | (mM⁻¹sec⁻¹):+ |
| SPIO | 50 nm | 12 nm | 1.2 | 247 |

*7T, 21° C.
:+7T, temp. not specified
**Chapon, C.; Franconi, F.; Lemaire, L.; Marescaux, L.; Legras, P.; Saint-Andre, J. P.; Denizot, B.; Le Jeune, J. J. Investigative Radiology 2003, 38, 141-146.

### REFERENCES

The following references are hereby incorporated by reference in their entirety.
Aaraki Y, Katoh T, Urabe M, Kishi Y. Ishizuka I, Fujiyama Y. The analysis of pyridylamino-dextran sulfate oligomers by high-performance liquid chromatography and a novel detection system for sulfated polysaccharides. Oncology Reports 2004;12(2):363-367.
Bowen C V, Zhang X, Saab G, Gareau P J, Rutt B K. Application of the static dephasing regime theory to superparamagnetic iron-oxide loaded cells. Magnetic Resonance in Medicine 2002;48(1):52-61.
Brown B G, Zhao X-Q, Sacco D E, Albers J J. Lipid lowering and plaque regression: New insights into prevention of plaque disruption and clinical events in coronary disease. Circulation 1993;87(6):1781-1791.
Bulte J W M, Kraitchman D L. Iron Oxide MR Contrast Agents for Molecular and Cellular Imaging. NMR in Biomedicine 2004;17:484-499.
Caravan P, Ellison J, McMurry T, Lauffer R. Gadolinium(III) Chelates as MRI Contrast Agents: Structure, Dynamics, and Applications. Chemical Reviews 1999;99(9):2293-2351.
Chen J W, Pham W, Weissleder R, Bogdanov A, Jr. Human myeloperoxidase: A potential target for molecular MR imaging in atherosclerosis. Magnetic Resonance in Medicine 2004;52(5):1021-1028.
Choudhury R P, Fuster V, Badimon J J, Fisher E A, Fayad Z A. MRI and characterization of atherosclerotic plaque: Emerging applications and molecular imaging. Arteriosclerosis Thrombosis & Vascular Biology 2002;22(7):1065-1074.
de Boer O J, Van Der Wal A C, Teeling P, Becker A E. Leucocyte recruitment in rupture prone regions of lipid-rich plaques: A prominent role for neovascularizaiton? Cardiovascular Research 1999;41(2):443-449.
de Winther M P J, van Dijk K W, Havekes L M, Hofker M H. Macrophage scavenger receptor class A: A multifunctional receptor in atherosclerosis. Arteriosclerosis Thrombosis & Vascular Biology 2000;20(2):290-297.
Dejager S, Mietus-Snyder M, Pitas R E. Oxidized low density lipoproteins bind to the scavenger receptor expressed by rabbit smooth muscle cells and macrophages. Arteriosclerosis & Thrombosis 1993;13(3):371-378.
Dodd S J, Williams M, Suhan J P, Williams D S, Koretsky A P, Ho C. Detection of single mammalian cells by high-resolution magnetic resonance imaging. Biophysical Journal 1999;76(1 PART A):103-109.
Euliss L E, Grancharov S G, O'Brien S, Deming T J, Stucky G D, Murray C B, Held G A. Cooperative Assembly of Magnetic Nanoparticles and Block Copolypeptides in Aqueous Media. Nano Letters 2003;3(11):1489-1493.
Flacke S, Fischer S, Scott M J, Fuhrhop R J, Allen J S, McLean M, Winter P, Sicard G A, Gaffney P J, Wickline S A, Lanza G M. Novel MRI contrast agent for molecular imaging of fibrin: Implications for detecting vulnerable plaques. Circulation 2001;104(11):1280-1285.
Gittins D I, Caruso F. Multilayered Polymer Nanocapsules Derived from Gold Nanoparticle Templates. Advanced Materials 2000;12(24):1947-1949.
Gittins D I, Caruso F. Tailoring the Polyelectrolyte Coating of Metal Nanoparticles. Journal of Physical Chemistry B 2001;105(29):6846-6852.
Glass C K, Witztum J L. Atherosclerosis: The road ahead. Cell 2001;104(4):503-516.
Heart Disease and Stroke Statistics—2005 Update, American Heart Association: American Heart Association; 2005. 1-63 p.
Heinecke J W. Oxidants and antioxidants in the pathogenesis of atherosclerosis: Implications for the oxidized low density lipoprotein hypothesis. Atherosclerosis 1998;141(1):1-15.
Herfkens R J, Higgins C B, Hricak H, Lipton M J, Crooks L E, Sheldon P E, Kaufman L. Nuclear Magnetic Resonance Imaging of Atherosclerotic Disease. Radiology 1983;148(1):161-166.
Heyn C, Bowen C V, Rutt B K, Foster P J. Detection Threshold of Single SPIO-Labeled Cells with FIESTA. Magnetic Resonance in Medicine 2005;53:312-320.
Jaffer F A, Weissleder R. Seeing within: Molecular imaging of the cardiovascular system. Circulation Research 2004;94(4):433-445.
Klotz M, Ayral A, Guizard C, Menager C, Cabuil V. Silica Coating on Colloidal Maghemite Particles. Journal of Colloid and Interface Science 1999;220:357-361.
Koenig S H, Kellar K E. Theory of 1/T-1 and 1/T-2 NMRD profiles of solutions of magnetic nanoparticles. Magnetic Resonance in Medicine 1995;34(2):227-233.
Koenig S H, Kellar K E, Fujii D K, Gunther W H H, Briley-Saebo K, Spiller M. Three Types of Physical Measurements Needed to Characterize Iron Oxide Nanoparticles for MRI and MRA: Magnetization, Relaxometry, and Light Scattering. Academic Radiology 2002;9 supplement 1(supplement 1):S5-S10.
Kooi M E, Cappendijk V C, Cleutjens K B J M, Kessels A G H, Kitslaar P J E H M, Borgers M, Frederik P M, Daemen M J A P, van Engelshoven J M A. Accumulation of ultrasmall superparamagnetic particles of iron oxide in human atherosclerotic plaques can be detected by in vivo magnetic resonance imaging. Circulation 2003;107(19):2453-2458.
Kramer C M, Cerilli L A, Hagspiel K, DiMaria J M, Epstein F H, Kern J A. Magnetic resonance imaging identifies the fibrous cap in atherosclerotic abdominal aortic aneurysm. Circulation 2004;109(3):1016-1021.
Lauffer R B. Paramagnetic metal complexes as water proton relaxation agents for NMR imaging: theory and design. Chemical Reviews (Washington, D.C., United States) 1987;87(5):901-927.
Libby P, Aikawa M. Stabilization of atherosclerotic plaques: New mechanisms and clinical targets. Nature Medicine 2002;8(11):1257-1262.
Lu Y. Yin Y, Mayers B T, Xia Y. Modifying the Surface Properties of Superparamagnetic Iron Oxide Nanoparticles through a Sol-Gel Approach. Nano Letters 2002;2(3):183-186.
Nagae T, Louie A Y, Aizawa K, Ishimaru S, Wilson S E. Selective targeting and photodynamic destruction of intimal hyperplasia by scavenger-receptor mediated protein-chlorin e6 conjugates. Journal of Cardiovascular Surgery 1998;39(6):709-715.
Netz R R, Joanny J-F. Complexation between a Semiflexible Polyelectrolyte and an Oppositely Charged Sphere. Macromolecules 1999;32(26):9026-9040.
Nitin N, LaConte L E W, Zurkiya O, Hu X, Bao G. Functionalization and peptide-based delivery of magnetic nanoparticles as an intracellular MRI contrast agent. Journal of Biological Inorganic Chemistry 2004;9(6):706-712.
O'Brien E R, Garvin M R, Dev R, Stewart D K, Hinohara T, Simpson J B, Schwartz S M. Angiogenesis in human coronary atherosclerotic plaques. American Journal of Pathology 1994;145(4):883-894.
Palmacci S, Josephson L; Advanced Magnetics, Inc. (Cambridge, Mass.), assignee. Synthesis of Polysaccharide Covered Superparamagnetic Oxide Colloids.

U.S. Pat. No. 5,262,176. 1993 Nov. 16, 1993.
Paul K G, Frigo T B, Groman J Y, Groman E V. Synthesis of Ultrasmall Superparamagnetic Iron Oxides Using Reduced Polysaccharides. Bioconjuqate Chemistry 2004;15(2):394-401.
Perez J M, Simeone F J, Tsourkas A, Josephson L, Weissleder R. Peroxidase Substrate Nanosensors for MR Imaging. Nano Letters 2004;4(1):119-122.
Platt N, Gordon S. Is the class A macrophage scavenger receptor (SR-A) multifunctional? The mouse's tale. Journal of Clinical Investigation 2001;108(5):649-654.
Podrez E A, Schmitt D, Hoff H F, Hazen S L. Myeloperoxidase-generated reactive nitrogen species convert LDL into an atherogenic form in vitro. Journal of Clinical Investigation 1999;103(11):1547-1560.
Pratten M K, Lloyd J B. Pinocytosis and Phagocytosis the Effect of Size of a Particulate Substrate on Its Mode of Capture by Rat Peritoneal Macrophages Cultured in-Vitro. Biochimica et Biophysica Acta 1986;881(3):307-313.
Roch A, Muller R N, Gillis P. Theory of Proton Relaxation Induced by Superparamagnetic Particles. Journal of Chemical Physics 1999;110(11):5403-5411.
Rogers W J, Basu P. Factors Regulating Macrophage Endocytosis of Nanoparticles: Implications for Targeted Magnetic Resonance Plaque Imaging. Atherosclerosis 2005;178:67-73.
Ross R. The pathogenesis of atherosclerosis: A perspective for the 1990s. Nature (London) 1993;362(6423):801-809.
Ross R. Atherosclerosis: An inflammatory disease. New England Journal of Medicine 1999;340(2):115-126.
Ruehm S G, Corot C, Vogt P, Kolb S, Debatin J F. Magnetic resonance imaging of atherosclerotic plaque with ultrasmall superparamagnetic particles of iron oxide in hyperlipidemic rabbits. Circulation 2001;103(3):415-422.
Sakai M, Kobori S, Miyazaki A, Horiuchi S. Macrophage proliferation in atherosclerosis. Current Opinion in Lipidology 2000;11(5):503-509.
Schmitz S A, Coupland S E, Gust R, Winterhalter S, Wagner S, Kresse M, Semmier W, Wolf K-J. Superparamagnetic iron oxide-enhanced MRI of atherosclerotic plaques in Watanabe hereditable hyperlipidemic rabbits. Investigative Radiology 2000;35(8):460-471.
Schmitz S A, Taupitz M, Wagner S, Coupland S E, Gust R, Nikolova A, Wolf K J. Iron-oxide-enhanced magnetic resonance imaging of atherosclerotic plaques: Postmortem analysis of accuracy, inter-observer agreement, and pitfalls. Investigative Radiology 2002;37(7):405-411.
Schmitz S A, Taupitz M, Wagner S, Wolf K-J, Beyersdorff D, Hamm B. Magnetic resonance imaging of atherosclerotic plaques using superparamagnetic iron oxide particles. Journal of Magnetic Resonance Imaging 2001;14(4):355-361.
Sun S, Zeng H, Robinson D B, Raoux S, Rice P M, Wang S X, Li G. Monodisperse MFe2O4 (M=Fe, Co, Mn) Nanoparticles. Journal of the American Chemical Society 2004;126(1):273-279.
Thomassen T, Wiggen U N, Gundersen H G, Fahivik A K, Aune O, Klaveness J. Structure Activity Relationship of Magnetic Particles as MR Contrast Agents. Magnetic Resonance Imaging 1991;9(2):255-258.
Toussaint J-F, Lamuraglia G M, Southern J F, Fuster V, Kantor H L. Magnetic resonance images lipid, fibrous, calcified, hemorrhagic, and thrombotic components of human atherosclerosis in vivo. Circulation 1996;94(5):932-938.
Toussaint J-F, Southern J F, Fuster V, Kantor H L. T-2-weighted contrast for NMR characterization of human atherosclerosis. Arteriosclerosis Thrombosis & Vascular Biology 1995;15(10):1533-1542.
Wilhelm C, Billotey C, Roger J, Pons J N, Bacri J C, Gazeau F. Intracellular uptake of anionic superparamagnetic nanoparticles as a function of their surface coating. Biomaterials 2003;24(6):1001-1011.
Winter P M, Caruthers S D, Yu X, Song S-K, Chen J, Miller B, Bulte J W M, Robertson J D, Gaffney P J, Wickline S A, Lanza G M. Improved molecular imaging contrast agent for detection of human thrombus. Magnetic Resonance in Medicine 2003a;50(2):411-416.
Winter P M, Morawski A M, Caruthers S D, Fuhrhop R W, Zhang H, Williams T A, Allen J S, Lacy E K, Robertson J D, Lanza G M, Wickline S A. Molecular imaging of angiogenesis in early-stage atherosclerosis with alphavbeta3-integrin-targeted nanoparticles. Circulation 2003b;108(18):2270-2274.
Yancy A D, Olzinski A R, Hu T C-C, Lenhard S C, Aravindhan K, Gruver S M, Jacobs P M, Willette R N, Jucker B M. Differential Uptake of Ferumoxotran-10 and Ferumoxytol, Ultrasmall Superparamagnetic Iron Oxide Contrast Agents in Rabbit: Critical Determinants of Atherosclerotic Plaque Labeling. Journal of Magnetic Resonance Imaging 2005;21:432-442.
Yung K-T. Empirical models of transverse relaxation for spherical magnetic perturbers. Magnetic Resonance Imaging 2003;21:451-463.

We claim:

1. A method of imaging a macrophage, the method comprising:
contacting a macrophage with a detection agent, wherein the detection agent comprises:
a detectable nanoparticle core;
a coating; and
a receptor binding moiety, wherein the receptor binding moiety binds to a receptor on a macrophage; and
detecting said agent to thereby image said macrophage.

2. The method of claim 1, wherein the macrophage is in a mammal.

3. The method of claim 2, wherein the macrophage is in an artery.

4. The method of claim 3, wherein the macrophage is in an atherosclerotic plaque.

5. The method of claim 4, wherein the atherosclerotic plaque is in a human patient.

6. The method of claim 2, further comprising administering to a mammal a detectable amount of the detection agent.

7. The method of claim 6, wherein administering is by intravenous or intraarterial injection.

8. The method of claim 1, wherein the detecting is by magnetic resonance imaging.

9. The method of claim 8, wherein the nanoparticle core is a metal oxide or a doped semiconductor.

10. The method of claim 9, wherein the metal oxide is an iron oxide, a manganese oxide or a lanthanide oxide.

11. The method of claim 9, wherein the doped semiconductor is doped with a paramagnetic atom or a paramagnetic molecule.

12. The method of claim 1, wherein the nanoparticle core is detectable by fluorescence spectroscopy.

13. The method of claim 12, wherein the nanoparticle core is a CdS or a ZnS nanoparticle.

14. The method of claim 1, wherein the nanoparticle core has a dimension less than about 100 nm.

15. The method of claim 14, wherein the nanoparticle core has a dimension between about 1 nm and about 30 nm.

16. The method of claim 15, wherein the nanoparticle core has a dimension between about 4 nm and about 15 nm, or between about 8 nm and about 12 nm.

17. The method of claim 1, wherein the detection agent is also a therapeutic agent.

18. The method of claim 1, wherein the coating is a polymer coating.

19. The method of claim 1, wherein the coating is the receptor binding moiety.

20. The method of claim 1, wherein the receptor binding moiety is polyanionic.

21. The method of claim 20, wherein the coating is dextran sulfate.

22. The method of claim 20, wherein the coating is silica.

23. The method of claim 1, wherein the receptor binding moiety is covalently attached to a linker molecule attached to the nanoparticle core.

24. The method of claim 23, wherein the linker molecule is a polyethylene glycol derivative.

25. The method of claim 24, wherein the linker molecule has a first functional group capable of binding to the nanoparticle core and a reactive functional group for attachment to the receptor binding moiety.

26. The method of claim 25, wherein the receptor binding moiety is an anionic moiety.

27. The method of claim 26, wherein the receptor binding moiety is oxLDL, polyinosinic acid, fucoidan, dextran sulfate, or maleylated-BSA.

28. An imaging agent comprising:
a detectable nanoparticle core
a coating;
a receptor binding moiety; and
a secondary detection moiety.

29. The imaging agent of claim 28, wherein the core is detectable by magnetic resonance imaging and is an iron oxide, a manganese oxide, a lanthanide oxide or a semiconductor doped with a paramagnetic atom or molecule.

30. The imaging agent of claim 28, wherein the secondary detection moiety is a fluorescent detection moiety or a positron emitting detection moiety.

31. The imaging agent of claim 28, wherein the imaging agent is also a therapeutic agent.

32. The imaging agent of claim 31, wherein the secondary detection moiety comprises ⁶⁴Cu.

33. The imaging agent of claim 28, wherein the nanoparticle core is fluorescent, and is a CdS or a ZnS nanoparticle.

34. The imaging agent of claim 33, wherein the secondary detection moiety is a magnetic resonance imaging contrast agent.

35. The imaging agent of claim 33, wherein the secondary detection moiety is a PET detection moiety.

36. The imaging agent of claim 28, wherein the coating is a polymer coating.

37. The imaging agent of claim 28, wherein the coating is the receptor binding moiety.

38. The imaging agent of claim 28, wherein the receptor binding moiety is polyanionic.

39. The imaging agent of claim 38, wherein the coating is dextran sulfate.

40. The imaging agent of claim 38, wherein the coating is silica.

41. The imaging agent of claim 28, wherein the receptor binding moiety is covalently attached to a linker molecule attached to the nanoparticle core.

42. The imaging agent of claim 41, wherein the linker molecule is a polyethylene glycol derivative.

43. The imaging agent of claim 42, wherein the linker molecule has a first functional group capable of binding to the nanoparticle core and a reactive functional group for attachment to the receptor binding moiety.

44. The imaging agent of claim 43, wherein the receptor binding moiety is an anionic moiety.

45. The imaging agent of claim 44, wherein the receptor binding moiety is oxLDL, polyinosinic acid, fucoidan, dextran sulfate, or maleylated-BSA.

46. A composition for imaging comprising:
a detectable nanoparticle core;
a coating; and
a receptor-binding moiety, wherein the receptor-binding moiety is polyanionic and is selected from the group consisting of oxLDL, polyinosinic acid, fucoidan, dextran sulfate, and maleylated-BSA.

47. The composition of claim 46, wherein the core is detectable by magnetic resonance imaging and is an iron oxide, a manganese oxide, a lanthanide oxide or a semiconductor doped with a paramagnetic atom or molecule.

48. The composition of claim 46, wherein the coating is a polymer coating.

49. The composition of claim 46, wherein the coating is the receptor binding moiety.

50. The composition of claim 46, wherein the receptor binding moiety is covalently attached to a linker molecule attached to the nanoparticle core.

51. The composition of claim 50, wherein the linker molecule is a polyethylene glycol derivative.

52. The composition of claim 51, wherein the linker molecule has a first functional group capable of binding to the nanoparticle core and a reactive functional group for attachment to the receptor binding moiety.

53. The composition of claim 52, wherein the receptor binding moiety is an anionic moiety.

54. The composition of claim 53, wherein the receptor binding moiety is oxLDL, polyinosinic acid, fucoidan, dextran sulfate, or maleylated-BSA.
